(19) United States
(12) Patent Application Publication
(10) Pub. No.: US 2002/0119100 A1
(43) Pub. Date: Aug. 29, 2002

(54) Detection material for initial dental caries

(75) Inventors: Akane Okada, Tokyo (JP); Yoko Ishihara, Tokyo (JP); Eiichi Yoshii, Tokyo (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: GC Corporation, Tokyo (JP)

(21) Appl. No.: 10/017,595

(22) Filed: Dec. 18, 2001

(30) Foreign Application Priority Data
Dec. 20, 2000 (JP) 2000-387636

(51) Int. Cl. A61B 5/00; A61B 8/00; A61B 10/00

(52) U.S. Cl. 424/9.7

(57) ABSTRACT

To simply and accurately detect initial dental caries without impairing the aesthetics, the detection material for initial dental caries contains 0.001 to 5% by weight of at least one dye selected from fluorescein sodium, fluorescein potassium, dibromofluorescein sodium, and dibromofluorescein potassium compounded in a solvent. It is preferred that the solvent is one member selected from water, ethanol, glycerin, isobutyl alcohol, ethyleneglycol, diethyleneglycol, triethyleneglycol, acetone, and propylene glycol, or a mixed solution of two or more of these members.

### BACKGROUND OF THE INVENTION

1. Field of the Invention

The present invention relates to a detection material for initial dental caries, which is capable of simply and accurately detecting initial dental caries without impairing the aesthetics. 2. Description of the Related Art

About a half of the causes of losing teeth is considered to be dental caries, and hence, it is important to prevent the dental caries. However, with respect to the dental caries, demineralization gradually proceeds in an unseen portion under a surface layer of a tooth, and as a result, when a subjective symptom such as one that can be observed with naked eye has appeared, the dental caries often proceeds to such an extent that the conservation remedy is needed. In other words, what is important in preventing the dental caries is to find out the demineralization proceeding under the surface layer of the tooth at an initial stage as far as possible, thereby giving a person a guidance for brushing the subject portion or subjecting to a treatment for stopping the progress of the demineralization by applying a fluoride. At the initial stage of the dental caries, even if the conservation remedy were needed, the remedy could be simple, so that a risk of the secondary dental caries can be minimized.

The demineralization under the surface layer as initial dental caries of a tooth is non-cavitation-forming dental caries that is free from substantial defects, formed due to the matter that during long-term repetition of demineralization wherein calcium ions and phosphate ions elute out from teeth due to plaque bacteria-producing acids and remineralization as a phenomenon wherein the calcium ions and phosphate ions are again taken into the teeth, a balance of the both is broken, and the environment is inclined towards the demineralization side over a long period of time. Thereafter, when the symptoms proceeds to some extent, it is confirmed as a white spot on an enamel from the clinical standpoint. It is considered that so far as the plaque is eliminated, and the surface of the teeth is brought into contact with saliva over a long period of time as far as possible, the initial dental caries up to the presence of a white spot does not proceed to the dental caries accompanied by substantial defects. In addition, it is already confirmed that when a fluoride is applied to an diseased part, the remineralization is promoted, whereby the initial dental caries can be restored to an original sound teeth to some extent. Accordingly, the detection for the initial dental caries has become important more and more in preventing the dental caries.

As a method for detecting the initial dental caries, a method in which a site from which the plaque has been eliminated is dried and inspected using a dental explorer and a dental mirror is the main current. However, it is very difficult to confirm the initial dental caries before the presence of a white spot. Further, there may be present a white spot portion generated by other causes than the demineralization such as one seen in the case of enamel hypoplasia. Accordingly, its diagnosis relied on the experiences and lacked in accuracy. Besides, as the detection and diagnosis techniques for the initial dental caries, there is hitherto known a method for measuring an electric resistance value of a tooth. However, this method involved a problem from the standpoint of the practical use because the measured values are scattered according to the measurement conditions and differences among individuals. Further, there are disclosed methods using devices, for example, a method in which a laser light having a specific wavelength is irradiated, and a reflected light from the irradiated site is quantitated, whereby the degree of progress of the dental caries is evaluated (see Japanese Patent Laid-Open No. 337142/1993); a method using an infrared camera (see Japanese Patent Laid-Open No. 233758/1996; and a method in which the water content in an initial dental caries site is quantitated by an infrared light (see Japanese Patent Laid-Open No. 71092/1996). The devices to be used in these methods are each required to use a large-sized and expensive detector, and therefore, have not been generally spreaded.

### SUMMARY OF THE INVENTION

The present invention is aimed to provide a detection material for initial dental caries, which is capable of simply and accurately detecting initial dental caries without impairing the aesthetics.

In order to achieve the above-described aim, we, the present inventors made extensive and intensive investigations. As a result, they paid attention to the matters that when a specific dye-containing detection material for initial dental caries having been applied to a tooth is irradiated with a light by means of a visible light irradiator to be used during polymerization of a photo-polymerizable dental restoration material such as dental composite resins, the initial caries site can be particularly distinctly discriminated and detected by a fluorescence emitted by excitation of the dye and that since this specific dye is of a yellowish system, which is inconspicuous at the time of application in an oral cavity, the aesthetics is not impaired after completion of the detection for initial dental caries, leading to accomplishment of the invention.

Specifically, the detection material for initial dental caries according to the present invention is a detection material for initial dental caries comprising 0.001 to 5% by weight of at least one dye selected from fluorescein sodium, fluorescein potassium, dibromofluorescein sodium, and dibromofluorescein potassium compounded in a solvent. As the solvent, preferred is a solution of one member selected from water, ethanol, glycerin, isobutyl alcohol, ethylene glycol, diethylene glycol, triethylene glycol, acetone, and propylene glycol, or a mixed solution of two or more of these members.

### DETAILED DESCRIPTION OF THE INVENTION

The at least one dye to be used for the detection material for initial dental caries according to the present invention, which is selected from fluorescein sodium, fluorescein potassium, dibromofluorescein sodium, and dibromofluorescein potassium, has such a characteristic that it is excited by a light irradiated from a dental visible light irradiator to emit a fluorescence. Further, since these dyes are one kind of tar dyes used in foods, medicine and cosmetics, they are highly safe to living bodies. Of these dyes, fluorescein sodium and fluorescein potassium are dyes called “Yellow No. 210(1)” and “Yellow No. 201(2)”, respectively according to the Japanese Color Name and when compounded in the solvent, are colored yellow. Dibromofluorescein sodium and dibromofluorescein potassium are called “Orange No.201(1)” and “Orange No. 201(2)”, respectively and when compounded in the solvent, are colored orange. Since the dye used in the invention is a dye that, when compounded in the solvent, is colored from orange to yellow, it has such a characteristic that in the case where it is used within an oral cavity, it does not impair the aesthetics unlikely the conventional dyes that when compounded in the solvent, are colored red, blue, green, or a mixed color thereof.

The at least one dye selected from fluorescein sodium, fluorescein potassium, dibromofluorescein sodium, and dibromofluorescein potassium to be used for the detection material for initial dental caries according to the present invention is compounded in an amount of 0.001 to 5% by weight in the detection material for initial dental caries. When the amount of the dye is less than 0.001% by weight, even if it is excited by a light irradiated from a dental visible light irradiator to emit a fluorescent, it is difficult to confirm the initial dental caries on a tooth surface. On the other hand, when the dye is compounded in an amount exceeding 5% by weight, since the visual evaluation is possible without irradiation with a light, the aesthetics is impaired. The amount of the dye to be compounded is preferably 0.001 to 0.5% by weight, and more preferably 0.01 to 0.09% by weight.

As the solvent that is used for the detection material for initial dental caries according to the present invention, employable widely are those solvents generally used in medicine, industries, and the like. Examples include water, ethanol, n-propanol, 2-butyl alcohol, isobutyl alcohol, n-amyl alcohol, isoamyl alcohol, ethylene glycol, 2-methoxyethanol, diethylene glycol, triethylene glycol, tetraethylene glycol, polyethylene glycol, propylene glycol, dipropylene glycol, polypropylene glycol, trimethylene glycol, 1,2-butanediol, 1,3-butanediol, 2,3-butanediol, 1,4-butanediol, 1,5-pentanediol, ethylene glycol monomethyl ether, ethylene glycol monomethyl ether acetate, ethylene glycol monoethyl ether, ethylene glycol diethylether, ethyleneglycolmonoethyletheracetate, ethylene glycol isopropyl ether, ethylene glycol monobutyl ether, ethylene glycol dibutyl ether, ethylene glycol monoacetate, ethylene glycol diacetate, diethylene glycol monomethyl ether, diethylene glycol monoethyl ether, diethylene glycol monoethyl ether acetate, diethylene glycol monobutyl ether, diethylene glycolmonobutyl ether acetate, diethylene glycol dimethyl ether, diethylene glycol methylethyl ether, diethylene glycol diethyl ether, diethylene glycol acetate, triethylene glycol monomethyl ether, triethylene glycol monoethyl ether, propylene glycol monomethyl ether, propylene glycol monoethyl ether, dipropylene glycol monomethyl ether, dipropylene glycol monoethyl ether,ee tripropylene glycol monomethyl ether, glycerin, tetrahydrofuran, dimethylformamide, dioxane, acetone, and dimethoxyethane. These solvents may be used singly or in admixture of two or more thereof. Of these, preferred are water, ethanol, glycerin, isobutyl alcohol, ethylene glycol, diethylene glycol, triethylene glycol, acetone, and propylene glycol, which are low in detrimental properties to human bodies.

As a method for using the detection material for initial dental caries according to the present invention, general is a method in which a tooth surface is first cleaned by brushing or other means and properly dried, and the detection material for initial dental caries is then applied by using a cotton ball or by dropping from a thin nozzle-equipped container. At this time, when the detection material for initial dental caries is compounded with a thickener as described later to increase its viscosity to 50 to 2,000 mPa·s (at 25° C.), thereby forming a gel, in the case where the resulting detection material for initial dental caries is applied to the tooth surface using a cotton ball, etc., it hardly sags, or by attaching it to a tooth brush and polishing the tooth surface, both the cleaning of the tooth surface and the application of the detection material for initial dental caries can be carried out simultaneously. Thus, such is preferred. Further, in the case where a mixed solution of water and ethanol is used as the solvent to be used for the detection material for initial dental caries according to the present invention, such is suitable for a method for letting a person hold it in his (or her) mouth and disgorge it.

After application of the detection material for initial dental caries, the tooth is allowed to stand for a necessary period of time although it varies depending on the solvent to be used, and then washed with water. Thereafter, the resulting tooth is irradiated with a light by a visible light irradiator with a wavelength of 320 to 700 nm, which is generally used in the dentistry, and a site emitting a fluorescence is observed during the light irradiation, whereby the presence or absence of the initial dental caries or its state and place are diagnosed. Example of light source which can be used include a chemical lamp, a carbon lamp, a methal halide lamp, a fluorescent lamp, a tangsten lamp, a xenon lamp, LED lamp and an argon ion laser. The thus confirmed initial dental caries portion is subjected to a treatment of giving a guidance for brushing or application of a fluoride, and the like depending on the symptoms.

Examples of the thickener that is used for the detection material for initial dental caries according to the present invention include synthetic additives such as sodium alginate, propylene glycol alginate, sodium carboxymethyl cellulose, calcium carboxymethyl cellulose, sodium carboxymethyl starch, sodium starch phosphate, sodium polyacrylate, methyl cellulose, hydroxypropyl cellulose, and polyvinylpyrrolidone; natural thickeners such as cyamoposis gum, Carob bean gum, Tara gum, Tamarind seed gum, gum arabic, tragacanth gum, Karaya gum, alginic acid, carrageenan, xanthan gum, gellan gum, curdlan, chitin, chitosan, and chitosamine; and inorganic thickeners such as calcium carbonate, calcium silicate, silica powder, amorphous hydrous silicate, and hydrophobic silica. As a result of experiments, it is already confirmed that an appropriate viscosity by the thickener is preferably in a range of 50 to 2,000 mPa·s (at 25° C.). In order to obtain this viscosity, the compounding amount of the thickener varies depending on the kind of the thickener. For example, when sodium carboxymethyl cellulose having a large thickening effect, or the like is used, the compounding amount may be about 0.5 to 4% by weight, and when methyl cellulose or the like is used, the compounding amount is required to be 10 to 30% by weight. That is, the compounding amount of the thickener is determined individually every thickener. Besides, as a matter of course, the detection material for initial dental caries according to the present invention may contain usual additives such as sweeteners, flavors, and preservatives.

Examples of the detection material for initial dental caries according to the present invention will be described below. It should not be construed that the invention is limited thereto.

### EXAMPLES 1 to 6

### Comparative Examples 1 to 2

### Preparation of Demineralized Teeth

After extirpation of pulp, bovine anterior teeth were placed in a silicone-made container having a diameter of 25 mm and a height of 15 mm such that the surface at the lip side was disposed upwardly, and invested by an acrylic resin. After curing the acrylic resin, the silicone-made container was removed, and the surface at the lip side was polished until an enamel had appeared. The resulting surface was provided as a test tooth surface. A circle having a diameter of 5 mm was set up on the test tooth surface, and the other exposed portion was covered by a nail varnish, followed by immersing in a Streptococcus sobrinus solution (1×10⁵ CFU/mL) at 37° C. for 24 hours and then washing with distilled water. There was thus prepared a demineralized teeth. Further, non-demineralized bovine anterior teeth were prepared as healthy teeth in the same manner except for not immersing them in a Streptococcus sobrinus solution.

### Detection for Initial Dental Caries Site

Detection materials for initial dental caries each having the compounding formulation as shown in Table 1 were prepared, and the initial dental caries was detected in the order as described below.

(1) The test tooth surface is washed by a tooth brush and with distilled water and then dried by air.

(2) The detection material for initial dental caries is applied on the test tooth surface using a cotton ball.

(3) Ten seconds after the application, the applied test tooth surface is washed with distilled water.

(4) The test tooth surface is evaluated on whether or not the dye is recognized on the sound site and the initial dental caries site, while irradiating with a light using a commercially available dental visible light irradiator (a trade name: Labolight LVII, manufactured by GC Corporation). At this time, the case where the dyeing to the sound teeth and the light irradiation were not carried out was also evaluated.

The results obtained are shown in Table 1. Further, in Comparative Example 1, used was a propylene glycol solution containing 0.1% by weight of acidic rhodamine as used for the conventional detection materials of dental caries; and in Comparative Example 2, used was a propylene glycol solution containing 0.1% by weight of a yellow dye (Tartrazine) that does not emit a fluorescence upon irradiation with the light by a visible light irradiator. Incidentally, the symbol “−” given in Table 1 means that the recognition could not be substantially made.

TABLE 1

| Component | Example 1 | Example 2 | Example 3 | Example 4 | Example 5 | Example 6 | Comparative Example 1 | Comparative Example 2 |
|---|---|---|---|---|---|---|---|---|
| Fluorescein sodium | 0.07 | | 0.05 | | 0.2 | | Propylene glycol solution | Propylene glycol solution |
| Fluorescein potassium | | 0.08 | | | | 0.3 | containing 0.1% by | containing 0.1% by |
| Dibromofluorescein | | | 0.04 | | | | weight of acidic | weight of Tartrazine |
| sodium | | | | | | | rhodamine | |
| Dibromofluorescein | | | | 0.1 | | | | |
| potassium | | | | | | | | |
| Water | 99.93 | 69.92 | 69.91 | | 69.8 | 94.7 | | |
| Ethanol | | | 20 | | 30 | | | |
| Acetone | | 30 | | | | | | |
| Propylene glycol | | | | 99.9 | | | | |
| Thickener* | | | 10 | | | 5 | | |
| 24 hr.-demineralized | — | — | — | — | — | — | Distinctly recognized | Slightly recognized |
| teeth without light | | | | | | | | |
| radiation | | | | | | | | |
| 24 hr.-demineralized | Distinctly | Distinctly | Distinctly | Distinctly | Distinctly | Distinctly | Distinctly recognized | — |
| teeth with light | recognized | recognized | recognized | recognized | recognized | recognized | | |
| radiation | | | | | | | | |
| Sound teeth without | — | — | — | — | — | — | Slightly recognized | — |
| light irradiation | | | | | | | | |
| Sound teeth with light | — | — | — | — | — | — | Slightly recognized | — |
| irradiation | | | | | | | | |

*: Hydroxypropyl cellulose

As is clear from each of the Examples, it has been confirmed that the detection material for initial dental caries according to the present invention is capable of simply and accurately detecting initial dental caries and when not irradiated with a light, does not impair the aesthetics. On the other hand, the detection material of dental caries of Comparative Example 1, in which the dye other than the specific dyes used in the present invention is used, is incapable of carrying out accurate detection, since it was recognized as if even the sound teeth had slight initial dental caries. Further, the oral cavity is always colored to an unnatural color regardless of the light irradiation, and hence, there is a problem from the standpoint of the aesthetics. In the detection material for dental caries of Comparative Example 2, in which the yellow dye that is not excited by a light and does not emit a fluorescence, the recognition of the initial dental caries portion was difficult.

As described above in detail, the detection material for initial dental caries according to the present invention is a detection material of dental caries, which is capable of simply and accurately detecting initial dental caries without impairing the aesthetics, and is greatly valuable in contributing to the dental remedy.

While the present invention has been described in detail and with reference to specific embodiments thereof, it will be apparent to one skilled in the art that various changes and modifications can be made therein without departing from the spirit and scope thereof.

What is claimed is:

1. A detection material for initial dental caries comprising 0.001 to 5% by weight of at least one dye selected from fluorescein sodium, fluorescein potassium, dibromofluorescein sodium, and dibromofluorescein potassium compounded in a solvent.

2. The detection material for initial dental caries as claimed in claim 1, wherein the solvent is one member selected from water, ethanol, glycerin, isobutyl alcohol, ethylene glycol, diethylene glycol, triethylene glycol, acetone, and propylene glycol, or a mixed solution of two or more of these members.
